(19) United States
(12) Patent Application Publication
(10) Pub. No.: US 2024/0043855 A1
(43) Pub. Date: Feb. 8, 2024

(54) A HELPER PLASMID FOR TRANSFORMATION, A METHOD FOR PRODUCING A TRANSFORMANT USING THE SAME, AND A METHOD OF TRANSFORMATION

(71) Applicant: TOYOTA JIDOSHA KABUSHIKI KAISHA, Toyota-shi, Aichi-ken (JP)

(72) Inventor: Toru ONISHI, Toyota-shi (JP)

(73) Assignee: TOYOTA JIDOSHA KABUSHIKI KAISHA, Toyota-shi, Aichi-ken (JP)

(21) Appl. No.: 18/258,799

(22) PCT Filed: Dec. 12, 2021
(86) PCT No.: PCT/JP2021/047336

§ 371 Date: Jun. 21, 2023

(30) Foreign Application Priority Data
Dec. 23, 2020 (JP) 2020-213107

(51) Int. Cl. C12N 15/81; C12N 15/90

(52) U.S. Cl. CPC C12N 15/81; C12N 15/905

(57) ABSTRACT

This invention is intended to simply and efficiently produce a stable transformant comprising a gene of interest incorporated into the genome. By introducing linear nucleic acid fragments for gemone-introduction comprising a gene of interest and a helper plasmid for transformation comprising a pair of homologous recombination sequences to incorporate the fragment and functioning a counter selection marker, a host comprising the helper plasmid for transformation into which the linear nucleic acid fragment is incorporated. Results in death.

### TECHNICAL FIELD

The present invention relates to a helper plasmid for transformation that is used upon introduction of a gene of interest into a host, a method of producing a transformant using the helper plasmid for transformation, and a transformation method using the helper plasmid for transformation.

### BACKGROUND ART

In general, a technique of introducing a gene of interest into a host cell from the outside is referred to as transformation or gene recombination, and a cell into which the gene of interest is introduced is referred to as a transformant or a recombinant. By efficiently producing such a transformant utilizing a transformation technique, acceleration and/or efficiency of microbial metabolic engineering can be promoted, for example, utilizing a synthetic biological technique. Herein, the synthetic biological technique means a technique of promptly turning a cycle consisting of the designing, construction, evaluation, and learning of a production host. In synthetic biology involving the use of a yeast host, in particular, it is important to efficiently construct a host, namely, to efficiently produce a recombinant yeast.

Transformation using a yeast as a host is roughly classified into a method involving the use of a circular plasmid into which a gene of interest is incorporated and a method involving the use of a linear vector comprising a gene of interest. It is easy to introduce a gene of interest into a yeast using a circular plasmid, and a transformed yeast can be produced at a high efficiency of approximately 10⁻² (Non-Patent Literature 1). On the other hand, when a gene of interest is introduced into a yeast using a linear vector, it is necessary to incorporate the gene of interest into the genome via homologous recombination. Thus, a transformed yeast can be produced only at an efficiency of approximately 10⁻⁶ (Non-Patent Literature 2).

As described above, the method of introducing a gene of interest into a yeast using a circular plasmid is highly efficient. However, such a circular plasmid may be detached in some case, and thus, a stable recombinant yeast cannot be produced. On the other hand, in the method of introducing a gene of interest into a yeast using a linear vector, the gene of interest is stably incorporated into the genome. However, as described above, this method is not considered to be highly efficient.

In order to improve the efficiency of introducing a gene of interest into the genome, known is a technique, in which the target sequence of target-specific endonuclease such as homing endonuclease has previously been introduced into a predetermined introduction site in the genome, and then, the double strands at the site have previously been cleaved (Non-Patent Literature 2). Moreover, also known is a technique, in which the double strands of a predetermined introduction site in the genome have previously been cleaved by applying a technique of cleaving any given nucleotide sequence, such as CRISPR-Cas9 or TALEN, instead of the target-specific endonuclease (Non-Patent Literature 3). Hence, it is possible to improve homologous recombination efficiency to approximately 10⁻² to 10⁻¹ by previously cleaving the double strands at the site into which a gene of interest is to be introduced.

However, in these methods of improving the efficiency of introducing a gene of interest, it has been necessary to previously introduce an endonuclease target sequence into a predetermined introduction site in the genome, or it has been necessary to produce guide RNA or the like corresponding to the target site. Thus, these methods of improving the efficiency of introducing a gene of interest are complicated, and require various steps, in addition to production of a DNA fragment for homologous recombination containing a gene of interest and the subsequent transformation using the produced DNA fragment.

In addition, Patent Literature 1 discloses a plasmid comprising a selection marker having an intron configured to sandwich a homing endonuclease recognition sequence with telomere seed sequences. In the case of the plasmid disclosed in Patent Literature 1, as a result of the expression of the homing endonuclease, the circular plasmid can be converted to linear molecules and can be stably present because of the telomere seed sequence at the terminus.

### CITATION LIST

### Patent Literature

PTL 1: US 2016/0017344

### Non Patent Literature

NPL 1: Gietz, R. D., et al., “High-efficiency yeast transformation using the LiAc/SS carrier DNA/PEG method,” Nature Protocols, 2, 2007: 31-34
NPL 2: Storici, F., et al., “Chromosomal site-specific double-strand breaks are efficiently targeted for repair by oligonucleotides in yeast,” Proc. Natl. Acad. Sci., U.S.A., 100, 2003: 14994-14999
NPL 3: DiCarlo, J. E., et al., “Genome engineering in Saccharomyces cerevisiae using CRISPR-Cas systems,” Nucleic Acids Res., 41, 2013: 4336-4343

### SUMMARY OF INVENTION

### Technical Problem

However, all of the aforementioned methods have been problematic in that a stable transformant, in which a gene of interest is incorporated into the genome, cannot be simply and efficiently produced according to the methods. Under the aforementioned circumstances, it is an object of the present invention to provide a method of producing a transformant capable of simply and efficiently producing a stable transformant, in which a gene of interest is incorporated into the genome, a transformation method, and a helper plasmid for transformation that can be used for such methods.

### Solution to Problem

The present invention that dissolves the aforementioned problem includes the following.
(1) A method of producing a transformant comprising:
a step of introducing: one or more linear nucleic acid fragments for gemone-introduction comprising a gene of interest to be introduced into a given site of a genome; and a helper plasmid for transformation comprising a pair of homologous recombination sequences to incorporate the linear nucleic acid fragment and a counter selection marker, into a host, wherein, in a state of introducing the linear nucleic acid fragment into the helper plasmid for transformation, a pair of homologous recombination sequences that undergoes homologous recombination between a region outside of the gene of interest and a given site of the genome and a pair of endonuclease target sequences at the outside of the pair of homologous recombination sequences are provided; and
a step of selecting a transformant in which the gene of interest is incorporated into the given site of the host genome, and in which the gene of interest is expressed,
wherein the counter selection marker functions to induce the death of a host comprising the helper plasmid for transformation into which the linear nucleic acid fragment is incorporated.
(2) The method of producing a transformant according to (1), wherein the helper plasmid for transformation comprises: a pair of homologous recombination sequences that undergoes homologous recombination with regions outside of a gene of interest in the linear nucleic acid fragment for gemone-introduction; and a pair of endonuclease target sequences provided on the other side of a site into which the linear nucleic acid fragment for gemone-introduction is incorporated via the homologous recombination sequences.
(3) The method of producing a transformant according to (1), wherein the linear nucleic acid fragment for gemone-introduction comprises: the pair of homologous recombination sequences to be incorporated into a given site of the genome at positions sandwiching the gene of interest; the pair of endonuclease target sequences outside of the pair of homologous recombination sequences; and the pair of homologous recombination sequences that undergoes homologous recombination with the helper plasmid for transformation outside of the pair of endonuclease target sequences.
(4) The method of producing a transformant according to (1), wherein the helper plasmid for transformation comprises a target-specific endonuclease gene specifically cleaving the double strands of the endonuclease target sequences in an expressible manner.
(5) The method of producing a transformant according to (4), wherein the target-specific endonuclease gene is a homing endonuclease gene.
(6) The method of producing a transformant according to (5), wherein the endonuclease target sequence is specifically recognized by homing endonuclease.
(7) The method of producing a transformant according to (4), wherein the helper plasmid for transformation comprises an inducible promoter regulating the expression of the target-specific endonuclease gene.
(8) The method of producing a transformant according to (1), wherein the plurality of the linear nucleic acid fragments consist of a 1^{st} linear nucleic acid fragment for gemone-introduction to the n^{th} linear nucleic acid fragment for gemone-introduction (n: an integer of 2 or greater), and a 3′ terminal sequence of the m^{th} linear nucleic acid fragment for gemone-introduction (m: an integer that satisfies the correlation: 1<=m<=n−1) comprises a sequence that undergoes homologous recombination with a 5′ terminal sequence of the m^{th}+1 linear nucleic acid fragment for gemone-introduction.
(9) A transformation method comprising:
a step of introducing: one or more linear nucleic acid fragments for gemone-introduction comprising a gene of interest to be introduced into a given site of a genome; and a helper plasmid for transformation comprising a pair of homologous recombination sequences to incorporate the linear nucleic acid fragment and a counter selection marker, into a host, wherein, in a state of introducing the linear nucleic acid fragment into the helper plasmid for transformation, a pair of homologous recombination sequences that undergoes homologous recombination between a region outside of the gene of interest and a given site of the genome and a pair of endonuclease target sequences at the outside of the pair of homologous recombination sequences are provided,
wherein the counter selection marker functions to induce the death of a host comprising the helper plasmid for transformation into which the linear nucleic acid fragment is incorporated.
(10) The transformation method according to (9), wherein the helper plasmid for transformation comprises: a pair of homologous recombination sequences that undergoes homologous recombination with regions outside of a gene of interest in the linear nucleic acid fragment for gemone-introduction; and a pair of endonuclease target sequences provided on the other side of a site into which the linear nucleic acid fragment for gemone-introduction is incorporated via the homologous recombination sequences.
(11) The transformation method according to (9), wherein the linear nucleic acid fragment for gemone-introduction comprises: the pair of homologous recombination sequences to be incorporated into a given site of the genome at positions sandwiching the gene of interest; the pair of endonuclease target sequences outside of the pair of homologous recombination sequences; and the pair of homologous recombination sequences that undergoes homologous recombination with the helper plasmid for transformation outside of the pair of endonuclease target sequences.
(12) The transformation method according to (9), wherein the helper plasmid for transformation comprises a target-specific endonuclease gene specifically cleaving the double strands of the endonuclease target sequences in an expressible manner.
(13) The transformation method according to (12), wherein the target-specific endonuclease gene is a homing endonuclease gene.
(14) The transformation method according to (13), wherein the endonuclease target sequence is specifically recognized by homing endonuclease.
(15) The transformation method according to (12), wherein the helper plasmid for transformation comprises an inducible promoter regulating the expression of the target-specific endonuclease gene.
(16) The transformation method according to (9), wherein the plurality of the linear nucleic acid fragments consist of a 1^{st} linear nucleic acid fragment for gemone-introduction to the n^{th} linear nucleic acid fragment for gemone-introduction (n: an integer of 2 or greater), and a 3′ terminal sequence of the m^{th} linear nucleic acid fragment for gemone-introduction (m: an integer that satisfies the correlation: 1<=m<=n−1) comprises a sequence that undergoes homologous recombination with a 5′ terminal sequence of the m^{th}+1 linear nucleic acid fragment for gemone-introduction.
(17) A helper plasmid for transformation capable of incorporating a linear nucleic acid fragment for gemone-introduction comprising a gene of interest to be introduced into a given site of a genome via homologous recombination, which comprises a pair of homologous recombination sequences that undergoes homologous recombination with regions outside of a gene of interest in the linear nucleic acid fragment for gemone-introduction; a pair of endonuclease target sequences provided on the other side of a site into which the linear nucleic acid fragment for gemone-introductions is incorporated via the homologous recombination sequences; and a counter selection marker.
(18) The helper plasmid for transformation according to (17), which comprises a target-specific endonuclease gene specifically cleaving the double strands of the endonuclease target sequences in an expressible state.
(19) The helper plasmid for transformation according to (18), wherein the target-specific endonuclease gene is a homing endonuclease gene.
(20) The helper plasmid for transformation according to (19), wherein the endonuclease target sequence is specifically recognized by homing endonuclease.
(21) The helper plasmid for transformation according to (18), which comprises an inducible promoter regulating the expression of the target-specific endonuclease gene.

This description includes part or all of the content as disclosed in the description and/or drawings of Japanese Patent Application No. 2020-213107, which is a priority document of the present application.

### Advantageous Effects of Invention

According to the method of producing a transformant of the present invention, the counter selection marker functions to induce death of a host in which a linear nucleic acid fragment for gemone-introduction having a gene of interest introduced therein has not been cleaved from the helper plasmid for transformation. Thus, a transformant in which a gene of interest is incorporated into a host genome can be efficiently produced.

According to transformation method of the present invention, in addition, the counter selection marker functions to induce death of a host in which a linear nucleic acid fragment for gemone-introduction having a gene of interest introduced therein has not been cleaved from the helper plasmid for transformation. Thus, excellent transformation efficiency can be achieved upon the production of a transformant in which a gene of interest is incorporated into the host genome.

With the use of the helper plasmid for transformation of the present invention, the counter selection marker functions to induce death of a host in which a linear nucleic acid fragment for gemone-introduction having a gene of interest introduced therein has not been cleaved from the helper plasmid for transformation. Thus, a transformant in which a gene of interest is incorporated into a host genome can be efficiently produced.

### BRIEF DESCRIPTION OF DRAWINGS

FIG. 1 is a configuration diagram schematically showing a mechanism of incorporating a gene of interest into a genome according to the method of producing a transformant and the transformation method of the present invention.

FIG. 2 is a configuration diagram schematically showing one configuration example of the helper plasmid for transformation according to the present invention.

FIG. 3 is a configuration diagram schematically showing the helper plasmid for transformation according to the present invention and the linear nucleic acid fragment for gemone-introduction.

FIG. 4 is a configuration diagram schematically showing another configuration example of the helper plasmid for transformation according to the present invention.

FIG. 5 is a configuration diagram schematically showing a mechanism of incorporating a plurality of genes of interest into a genome using the helper plasmid for transformation according to the present invention.

FIG. 6 is a configuration diagram schematically showing one configuration example of the linear nucleic acid fragment for gemone-introduction and the helper plasmid for transformation demonstrated as the second embodiment.

FIG. 7 is a configuration diagram schematically showing a mechanism of incorporating a gene of interest into a genome according to the method of producing a transformant and the transformation method demonstrated as the second embodiment.

FIG. 8 is a configuration diagram schematically showing a mechanism of incorporating a plurality of genes of interest into a genome according to the method of producing a transformant and the transformation method demonstrated as the second embodiment.

FIG. 9 is a configuration diagram schematically showing a scheme for amplifying the 3 types of linear nucleic acid fragments for gemone-introduction produced in the examples.

FIG. 10 is a configuration diagram schematically showing a scheme for amplifying the helper plasmid for transformation produced in the examples.

### DESCRIPTION OF EMBODIMENTS

Hereinafter, the present invention will be described in more detail using drawings and examples.

According to the method of producing a transformant and the transformation method according to the present invention (hereafter, these methods are collectively referred to as “the methods of the present invention”), a linear nucleic acid fragment for gemone-introduction having a gene of interest to be incorporated into a host genome and a helper plasmid for transformation capable of incorporating the linear nucleic acid fragment for gemone-introduction via homologous recombination and having a counter selection marker are introduced into a host. According to the methods of the present invention, the linear nucleic acid fragment for gemone-introduction is incorporated into the helper plasmid for transformation via homologous recombination. In the host, a gene of interest sandwiched with (or interposed between) a pair of homologous recombination sequences is cleaved by a given endonuclease, and the gene of interest is thus incorporated into the genome via homologous recombination with the host genome.

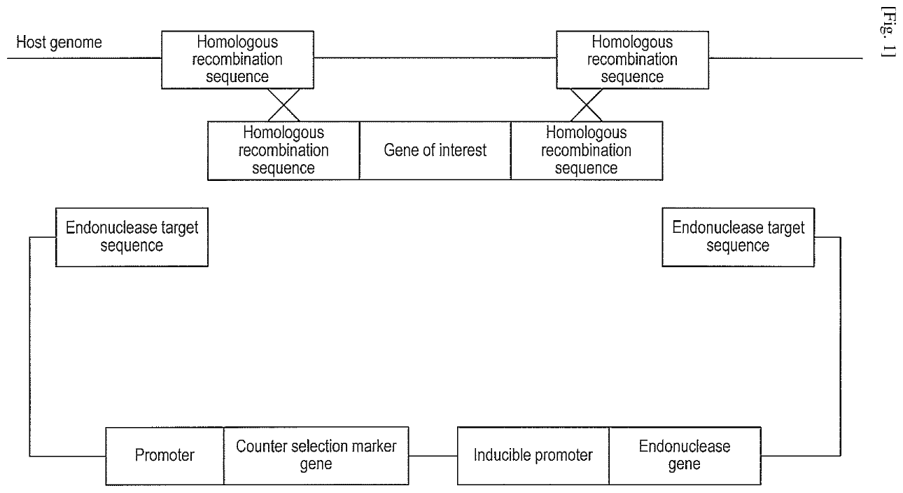

In this case, a gene of interest sandwiched with a pair of homologous recombination sequences is provided to be sandwiched with a pair of endonuclease target sequences, so that the gene of interest sandwiched with a pair of homologous recombination sequences can be cleaved by the endonuclease that specifically recognizes the endonuclease target sequences. As schematically shown in FIG. 1, specifically, a fragment cleaved from the plasmid with the aid of the endonuclease is configured to comprise, at its both ends, a pair of homologous recombination sequences and a gene of interest sandwiched with the pair of homologous recombination sequences. As a result of homologous recombination between the pair of homologous recombination sequences and the host genome, the gene of interest can be incorporated into the host genome.

The pair of endonuclease target sequences may be provided in the linear nucleic acid fragment for gemone-introduction to be introduced into the host or in the helper plasmid for transformation in advance. Alternatively, one of the pair of endonuclease target sequences may be provided in the linear nucleic acid fragment for gemone-introduction in advance, and the other may be provided in the helper plasmid for transformation in advance.

The term “counter selection marker” used herein refers to, for example, a gene that has functions of inducing cell death by being expressed in the cell, and capable of using as a marker. In this case, a cell comprising a counter selection marker is induced to die upon expression of the counter selection marker gene under particular conditions. In the presence of both a cell with the counter selection marker and a cell without the counter selection marker, accordingly, a cell that has grown under particular conditions can be selected as a cell without the selection marker.

A counter selection marker may be, for example, a gene that has functions of inducing cell death when gene expression is suppressed under particular conditions. When a counter selection marker functions, expression of a counter selection marker gene is suppressed under particular conditions, and the cell is induced to die. In the presence of both a cell with the counter selection marker and a cell without the counter selection marker, accordingly, a cell that has grown under particular conditions can be selected as a cell without the selection marker.

When an E. coli is used as a host, specifically, the sacB gene derived from Bacillus subtilis can be used as a counter selection marker. A sacB gene product, levansucrase, has activity of converting sucrose to levan. Gram-negative bacteria, such as Escherichia coli, are induced to die upon accumulation of levan in the periplasm layer. Thus, the sacB gene can be used as a counter selection marker.

Another example of a counter selection marker is a variant of an alpha-subunit of phenylalanyl tRNA synthetase (PheS). A PheS variant incorporates a phenylalanine analog, which is 4-chloro-D,L-phenylalanine. Thus, a cell in which a PheS variant is expressed is not capable of synthesizing a normal polypeptide. Because a normal polypeptide cannot be synthesized, a cell in which the PheS variant is expressed is induced to die in the presence of 4-chloro-D,L-phenylalanine. By introducing an amino acid analog into a biosynthesized protein molecule, as described above, functions thereof would be damaged, and the cell can be induced to die. A variant gene that can be used in such method can be used as a counter selection marker.

Another example of a counter selection marker is a thymidine kinase gene. The thymidine kinase gene converts 5-fluoro-2-deoxyuridine (5FU) into a toxic metabolite, 5-fluorodeoxyuridine-5′-monophosphate, and inhibits thymine biosynthesis by inhibiting a thymidylate synthase. Accordingly, a cell in which the thymidine kinase gene is expressed is induced to die upon inhibition of thymine biosynthesis in the presence of 5FU.

In addition, a temperature-sensitive variant gene of a replication origin of the pSC101 plasmid (RepA) can be used as a counter selection marker. By subjecting a cell that carries a plasmid comprising such temperature-sensitive variant gene to culture at temperature over 37 degrees C., growth of the cell is inhibited. By transforming a cell with the use of a plasmid comprising such temperature-sensitive variant gene and performing culture in the temperate range described above, a cell from which a plasmid is detached can be selectively grown.

Further, a toxin-antitoxin system can also be used as a counter selection marker. When an antitoxin gene is expressed, in general, cell death induced by expression of a toxin gene is inhibited. By inhibiting antitoxin gene expression, accordingly, effects achieved by toxin gene expression can be made apparent, and cell death can be induced. By designing an antisense RNA that targets the endogenous antitoxin gene as a nucleotide sequence complementary to a part of mRNA of the antitoxin gene and inducing antisense RNA in a condition-specific manner, for example, antitoxin gene translation can be inhibited. As a result, the cell death can be induced upon toxin gene expression.

### First Embodiment

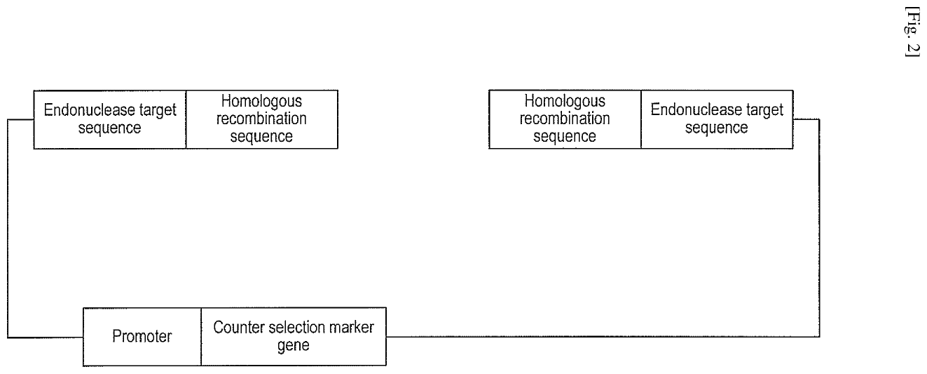

Hereafter, an embodiment in which a pair of endonuclease target sequences is provided in a helper plasmid for transformation is described. The helper plasmid for transformation according to the present invention comprises: as shown in FIG. 2, a pair of homologous recombination sequences to incorporate a linear nucleic acid fragment for gemone-introduction; a pair of endonuclease target sequences provided on the other side of a site into which the linear nucleic acid fragment for gemone-introduction is to be incorporated via the homologous recombination sequence; a promoter and a counter selection marker gene downstream of the promoter. In other words, when the site into which the linear nucleic acid fragment for gemone-introduction is to be incorporated is cleaved to be linearized, the helper plasmid for transformation comprises, at both ends, a pair of homologous recombination sequences, each of endonuclease target sequences subsequent to the homologous recombination sequences, respectively, the promoter and the counter selection marker gene downstream of the promoter.

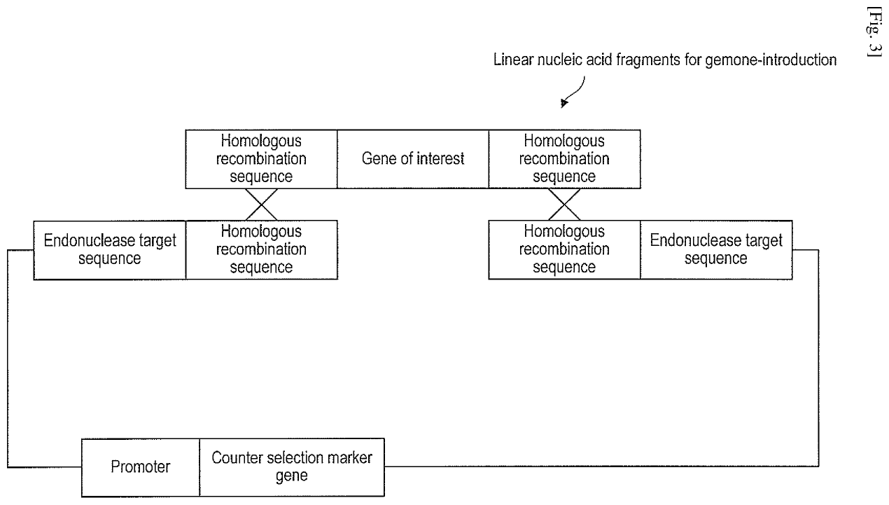

As shown in FIG. 3, the helper plasmid for transformation is capable of incorporating a linear nucleic acid fragment for gemone-introduction comprising a gene of interest by homologous recombination via the pair of homologous recombination sequences. The linear nucleic acid fragment for gemone-introduction comprises a gene of interest and a pair of homologous recombination sequences sandwiching the gene of interest. Upon homologous recombination occurring between the homologous recombination sequences of the linear nucleic acid fragment for gemone-introduction and the homologous recombination sequences of the helper plasmid for transformation, specifically, the linear nucleic acid fragment for gemone-introduction can be incorporated into the helper plasmid for transformation. Upon homologous recombination occurring between the homologous recombination sequence of the linear nucleic acid fragment for gemone-introduction and a given site in the genome, a gene of interest can be incorporated into the genome (see FIG. 1).

The term “a gene of interest” means a nucleic acid to be introduced into a host genome. Accordingly, such a gene of interest is not limited to a nucleotide sequence encoding a specific protein, and includes nucleic acids consisting of all types of nucleotide sequences, such as a nucleotide sequence encoding siRNA, etc., a nucleotide sequence of a transcriptional regulatory region that regulates the transcription period of a transcriptional product and the production amount thereof, such as a promoter or an enhancer, and a nucleotide sequence encoding transfer RNA (tRNA), ribosome RNA (rRNA), etc.

Furthermore, such a gene of interest is preferably incorporated into the above-described site in an expressible manner. In an expressible manner, a gene of interest is linked to a predetermined promoter and is then incorporated into the above-described site, so that the gene of interest can be expressed under the control of the promoter in a host organism.

In addition, a promoter and a terminator, and as desired, a cis element such as an enhancer, a splicing signal, a poly A addition signal, a selection marker, a ribosomal binding sequence (SD sequence), and the like can be linked to such a gene of interest. Examples of the selection marker include antibiotic resistance genes such as an ampicillin resistance gene, a kanamycin resistance gene, and a hygromycin resistance gene.

The term “a pair of homologous recombination sequences” means a pair of nucleic acid regions having homology to a certain region in a host genome. Such a pair of homologous recombination sequences of the linear nucleic acid fragment for gemone-introduction each cross with the host genome having homology to the homologous recombination sequences, so that a gene of interest sandwiched with the pair of homologous recombination sequences can be incorporated into the host genome. Accordingly, such a pair of homologous recombination sequences are not particularly limited to specific nucleotide sequences, and can be, for example, nucleotide sequences having high homology to the upstream region and the downstream region of a certain gene present in the host genome. In this case, if homologous recombination takes place between the linear nucleic acid fragment for gemone-introduction and the host genome, the gene is deleted from the host genome. As such, the success or failure of homologous recombination can be determined by observing a phenotype caused by the deletion of the gene.

For example, such a pair of homologous recombination sequences can be a region upstream of the coding region of an ADE1 gene associated with an adenine biosynthesis pathway, and a region downstream of the coding region of the ADE1 gene. In this case, if homologous recombination takes place between the pair of homologous recombination sequences of the linear nucleic acid fragment for gemone-introduction and the host genome, an intermediate metabolite of adenine, 5-aminoimidazole riboside, is accumulated, and a transformant is colored red due to the polymerized polyribosylaminoimidazole. Accordingly, by detecting this red color, it can be determined that homologous recombination has taken place between the pair of homologous recombination sequences of the linear nucleic acid fragment for gemone-introduction and the host genome.

Herein, the pair of homologous recombination sequences of the linear nucleic acid fragment for gemone-introduction has high sequence identity to the recombination region in the host genome, to such an extent that they can be homologously recombined (can cross) with each other. The identity between the nucleotide sequences of individual regions can be calculated using conventional sequence comparison software “blastn,” etc. The nucleotide sequences of individual regions may have an identity of 60% or more, and the sequence identity is preferably 80% or more, more preferably 90% or more, particularly preferably 95% or more, and the most preferably 99% or more.

Still further, such a pair of homologous recombination sequences may have the same length, or may each have different lengths. The lengths of such a pair of homologous recombination sequences of the linear nucleic acid fragment for gemone-introduction are not particularly limited, as long as they can undergo homologous recombination (crossing) with the genome. The length of each of the pair of homologous recombination sequences is, for example, preferably 0.1 kb to 3 kb, more preferably 0.5 kb to 3 kb, and particularly preferably 0.5 kb to 2 kb.

The helper plasmid for transformation according to the present invention comprises a pair of homologous recombination sequences used for incorporation of the linear nucleic acid fragment for gemone-introduction. It is sufficient if homologous recombination takes place between the homologous recombination sequence of the helper plasmid for transformation and the homologous recombination sequence of the linear nucleic acid fragment for gemone-introduction. The length of the homologous recombination sequence of the helper plasmid for transformation may be the same with or different from the length of the homologous recombination sequence of the linear nucleic acid fragment for gemone-introduction. The homologous recombination sequence of the helper plasmid for transformation has homology to the homologous recombination sequence of the linear nucleic acid fragment for gemone-introduction. For example, the length may be 30 b to 300 b, preferably 40 b to 200 b, and more preferably 50 b to 100 b.

In the helper plasmid for transformation according to the present invention, a pair of homologous recombination sequences used for incorporation of the linear nucleic acid fragment for gemone-introduction encompasses those that can undergo homologous recombination directly with a pair of homologous recombination sequences of the linear nucleic acid fragment for gemone-introduction and those that can undergo homologous recombination indirectly with a pair of homologous recombination sequences of the linear nucleic acid fragment for gemone-introduction via 1 or more linear nucleic acid fragments. Here, 1 or more linear nucleic acid fragments refer to one nucleic acid fragment or a nucleic acid fragment comprising a plurality of nucleic acid fragments linked to one another via homologous recombination, in which each comprise a sequence that can undergo homologous recombination with the homologous recombination sequence of the helper plasmid for transformation at one end and a sequence that can undergo homologous recombination with the homologous recombination sequence of the linear nucleic acid fragment for gemone-introduction at the other end.

The helper plasmid for transformation according to the present invention comprises endonuclease target sequences subsequent to the aforementioned pair of homologous recombination sequences. The term “endonuclease target sequence” means a nucleotide sequence recognized by endonuclease.

The endonuclease is not particularly limited, and it extensively means an enzyme having activity of recognizing a predetermined nucleotide sequence and cleaving double-stranded DNA. Examples of the endonuclease include restriction enzymes, homing endonuclease, Cas9 nuclease, meganuclease (MN), zinc finger nuclease (ZFN), and transcriptional activation-like effector nuclease (TALEN). Moreover, the term “homing endonuclease” includes both endonuclease encoded by an intron (with the prefix “I-”) and endonuclease included in an intein (with the prefix “PI-”). More specific examples of the homing endonuclease include I-Ceu I, I-Sce I, I-Onu I, PI-Psp I, and PI-Sce I. Besides, target sequences specifically recognized by these specific endonucleases, namely, endonuclease target sequences, are known, and a person skilled in the art could appropriately acquire such endonuclease target sequences.

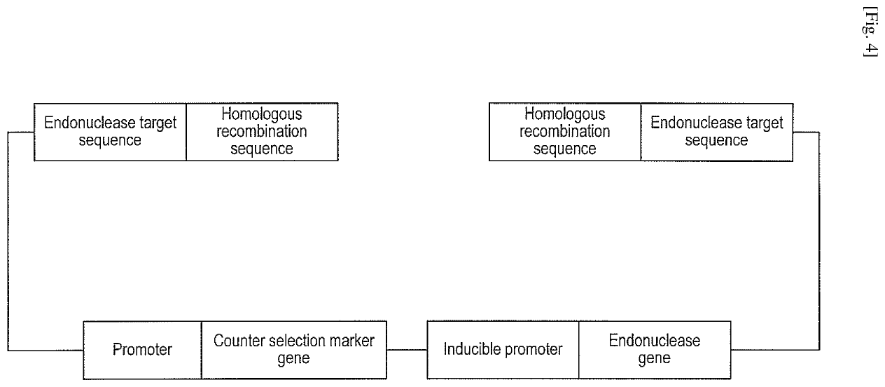

Moreover, as shown in FIG. 4, the helper plasmid for transformation according to the present invention may comprise an inducible promoter and an endonuclease gene. For the expression of an endonuclease gene, not only an inducible promoter, but also a consititutive expression promoter may be used.

This endonuclease gene encodes an enzyme having activity of specifically recognizing the aforementioned pair of endonuclease target sequences and cleaving the double strands. That is, examples of the endonuclease gene include a restriction enzyme gene, a homing endonuclease gene, a Cas9 nuclease gene, a meganuclease gene, a zinc finger nuclease gene, and a transcriptional activation-like effector nuclease gene.

The inducible promoter means a promoter having functions of inducing expression under specific conditions. Examples of the inducible promoter include, but are not particularly limited to, a promoter inducing expression in the presence of a specific substance, a promoter inducing expression under specific temperature conditions, and a promoter inducing expression in response to various types of stresses. The used promoter can adequately be selected depending on a host to be transformed.

Examples of the inducible promoter include galactose inducible promoters such as GAL1 and GAL10, Tet-on/Tet-off system promoters inducing expression with the addition or removal of tetracycline or a derivative thereof, and promoters of genes encoding heat shock proteins (HSP) such as HSP10, HSP60, and HSP90. In addition, as such an inducible promoter, a CUP1 promoter that activates with the addition of copper ions can also be used. Furthermore, when the host is a prokaryotic cell such as Escherichia coli, examples of the inducible promoter include a lac promoter inducing expression with IPTG, a cspA promoter inducing expression by cold shock, and an araBAD promoter inducing expression with arabinose.

Further, the method of controlling the expression of an endonuclease gene is not limited to a method involving the use of a promoter such as an inducible promoter or a consititutive expression promoter. For example, a method involving the use of DNA recombinase may be applied. An example of the method of turning the expression of a gene ON and OFF with the use of DNA recombinase may be a FLEx switch method (A FLEX Switch Targets Channelrhodopsin-2 to Multiple Cell Types for Imaging and Long-Range Circuit Mapping. Atasoy et al., The Journal of Neuroscience, 28, 7025-7030, 2008). According to the FLEx switch method, recombination to change the direction of a promoter sequence is caused by DNA recombinase, so that the expression of a gene can be turned ON and OFF.

On the other hand, the helper plasmid for transformation according to the present invention can be produced based on a conventional, available plasmid. Examples of such a plasmid include: YCp-type E. coli-yeast shuttle vectors, such as pRS413, pRS414, pRS415, pRS416, YCp50, pAUR112, and pAUR123; YEp-type E. coli-yeast shuttle vectors, such as pYES2 and YEp13; YIp-type E. coli-yeast shuttle vectors, such as pRS403, pRS404, pRS405, pRS406, pAUR101, and pAUR135; Escherichia coli derived plasmids (e.g., ColE-type plasmids, such as pBR322, pBR325, pUC18, pUC19, pUC118, pUC119, pTV118N, pTV119N, pBluescript, pHSG298, pHSG396, and pTrc99A; p15A-type plasmids, such as pACYC177 and pACYC184; and pSC101-type plasmids, such as pMW118, pMW119, pMW218, and pMW219); Agrobacterium-derived plasmids (e.g., pBI101); and Bacillus subtilis-derived plasmids (e.g., pUB110 and pTP5).

Moreover, the helper plasmid for transformation according to the present invention may further comprise a replication origin, an autonomously replicating sequence (ARS), and a centromere sequence (CEN). The helper plasmid for transformation comprises these elements, so that it can stably replicate after it has been introduced into a host cell. In addition, the helper plasmid for transformation according to the present invention may comprise a selection marker. The selection marker is not particularly limited, and examples of the selection marker include a drug resistance marker gene and an auxotrophic marker gene. The helper plasmid for transformation comprises these selection markers, so that a host cell into which the helper plasmid for transformation has been introduced can be efficiently selected.

By using the thus configured helper plasmid for transformation, a stable transformant in which a gene of interest is incorporated into the genome can be simply and efficiently produced. To produce a transformant, at the outset, a linear nucleic acid fragment for gemone-introduction comprising a gene of interest and a helper plasmid for transformation are introduced into a host cell in accordance with a conventional technique. In this case, the linear nucleic acid fragment for gemone-introduction is incorporated into the helper plasmid for transformation to result in a circular plasmid (see FIG. 3). Thereafter, as schematically shown in FIG. 1, the double stands of a pair of endonuclease target sequences are cleaved by endonuclease, and the linear nucleic acid fragment for gemone-introduction sandwiched with the pair of homologous recombination sequences is cleaved out. A pair of homologous recombination sequences in the thus cleaved linear nucleic acid fragment for gemone-introduction crosses with the homologous recombination sequences in the host genome, and the gene of interest is then incorporated into the genome. Thus, a stable transformant in which the gene of interest is incorporated into the genome can be produced.

Herein, the method of introducing the linear nucleic acid fragment for gemone-introduction comprising a gene of interest and the helper plasmid for transformation into a host cell is not particularly limited, and conventional methods, such as a calcium chloride method, a competent cell method, a protoplast or spheroplast method, or an electrical pulse method, can be adequately employed. When the helper plasmid for transformation comprises a selection marker, the host cell into which the helper plasmid for transformation has been introduced can then be selected using the selection marker.

In order to allow endonuclease to express under the control of an inducible promoter, in addition, conditions are adequately determined depending on the type of the inducible promoter. When a galactose inducible promoter such as GAL1 or GAL10 is used as such an inducible promoter, for example, galactose is added to a medium for use in the culture of the host cell into which the helper plasmid for transformation has been introduced, or the host cell is transferred to a galactose-containing medium and is then cultured, so that the expression of the endonuclease can be induced. When a promoter of a gene encoding a heat shock protein (HSP) is used as such an inducible promoter, heat shock is applied to the host cell into which the helper plasmid for transformation has been introduced at a desired timing during the culture of the host cell, so that the expression of the endonuclease can be induced at the desired timing.

Under the conditions in which the inducible promoter induces expression, a linear nucleic acid fragment for gemone-introduction and a helper plasmid for transformation may be introduced into a host cell and the endonuclease may then be expressed under the control of the inducible promoter. In such a case, it is not necessary to transfer the host cell to the conditions under which the inducible promoter induces expression. Thus, a transformant can be obtained more easily.

Furthermore, in the aforementioned helper plasmid for transformation, when the pair of homologous recombination sequences have high homology to the upstream region and the downstream region of a predetermined gene, the linear nucleic acid fragment for gemone-introduction comprising a gene of interest is incorporated into the genome via homologous recombination, and, at the same time, the predetermined gene is deleted from the genome. By observing a phenotype resulting from the deletion of the predetermined gene, accordingly, whether or not the linear nucleic acid fragment for gemone-introduction comprising a gene of interest has been incorporated into the genome can be determined. When an ADE1 gene is utilized as such a predetermined gene, for example, the ADE1 gene is deleted from the genome if the linear nucleic acid fragment for gemone-introduction comprising a gene of interest is incorporated into the genome. As a result, 5-aminoimidazole riboside is accumulated in the host, and a transformant is colored red due to the polymerized polyribosylaminoimidazole. By detecting this red color, accordingly, it can be determined that the linear nucleic acid fragment for gemone-introduction comprising a gene of interest has been incorporated into the genome of the host.

It is to be noted that, in the aforementioned example, the helper plasmid for transformation is configured to comprise an inducible promoter and an endonuclease gene, but that the helper plasmid for transformation according to the present invention may also be configured not to comprise such an inducible promoter and an endonuclease gene. In this case, an expression vector comprising an inducible promoter and an endonuclease gene may be prepared separately, and the expression vector may be introduced into a host cell together with the linear nucleic acid fragment for gemone-introduction comprising a gene of interest and the helper plasmid for transformation according to the present invention. Even in this case, in the host cell into which the expression vector comprising an inducible promoter and an endonuclease gene, the linear nucleic acid fragment for gemone-introduction, and the helper plasmid for transformation have been introduced, the endonuclease gene is expressed under the control of the inducible promoter, so that, as shown in FIG. 1, a linear nucleic acid fragment for gemone-introduction comprising a gene of interest sandwiched with a pair of homologous recombination sequences can be cleaved out, and a transformant in which the gene of interest is incorporated into the genome can be produced.

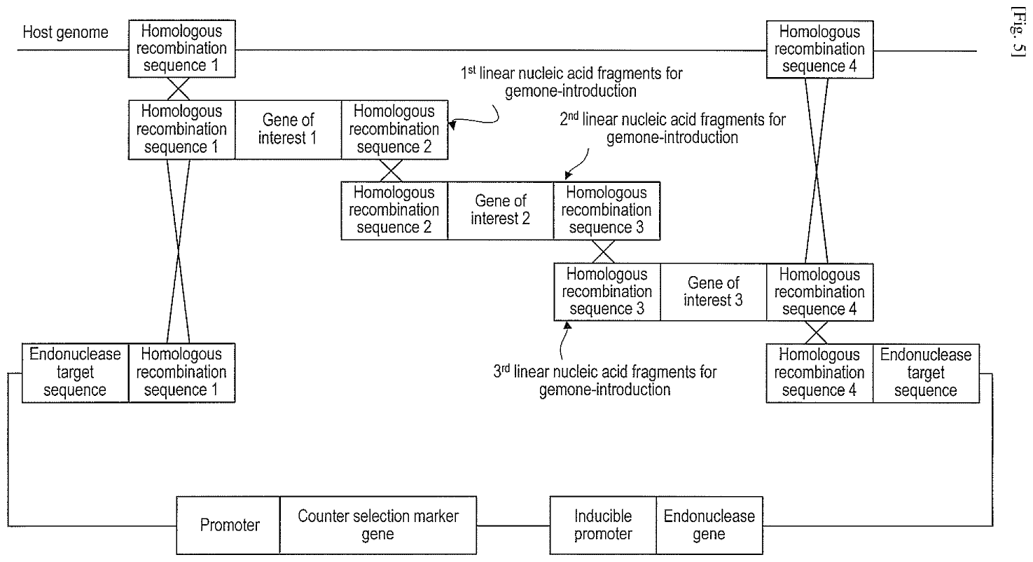

Meanwhile, with the use of the helper plasmid for transformation according to the present invention, a plurality of linear nucleic acid fragments for gemone-introduction can be arranged in series and incorporated into the host genome. A plurality of linear nucleic acid fragments for gemone-introduction are designated to be the 1^{st} linear nucleic acid fragment for gemone-introduction to the n^{th} linear nucleic acid fragment for gemone-introduction (n: an integer of 2 or greater). A 3′ terminal sequence of the m^{th} linear nucleic acid fragment for gemone-introduction (m: an integer that satisfies the correlation: 1<=m<=n−1 (i.e. m is greater than or equal to 1 and less than or equal to n−1)) and a 5′ terminal sequence of the m^{th}+1 linear nucleic acid fragment for gemone-introduction are to be homologous recombination sequences. Thus, the 1^{st} to the n^{th} linear nucleic acid fragments for gemone-introduction can be connected to one another in this order via homologous recombination. As shown in FIG. 5, in the case that the 1^{st} to the 3^{rd} linear nucleic acid fragment for gemone-introduction are incorporated into the host genome, for example, the 3′ terminal sequence of the 1^{st} linear nucleic acid fragment for gemone-introduction and the 5′ terminal sequence of the 2^{nd} linear nucleic acid fragment for gemone-introduction are to be the homologous recombination sequence 2, and the 3′ terminal sequence of the 2^{nd} linear nucleic acid fragment for gemone-introduction and the 5′ terminal sequence of the 3^{rd} linear nucleic acid fragment for gemone-introduction are to be the homologous recombination sequence 3. Thus, the 1^{st} to the 3^{rd} linear nucleic acid fragments for gemone-introduction can be connected to one another in this order via homologous recombination. A fragment comprising the 1^{st} the 3^{rd} linear nucleic acid fragment for gemone-introduction connected to one another is incorporated into the helper plasmid for transformation and then into the host genome via homologous recombination between the helper plasmid for transformation and the host genome via the homologous recombination sequence 1 and the homologous recombination sequence 4.

In order to arrange a plurality of linear nucleic acid fragments for gemone-introduction in series via homologous recombination, homologous recombination sequences are provided between linear nucleic acid fragments for gemone-introduction adjacent to each other. It is sufficient if homologous recombination takes place between homologous recombination sequences of the linear nucleic acid fragments for gemone-introduction adjacent to each other. The lengths of the homologous recombination sequences of the linear nucleic acid fragments for gemone-introduction adjacent to each other may be the same with or different from each other. Such a homologous recombination sequence is a nucleotide sequence having homology to the homologous recombination sequence of the linear nucleic acid fragments for gemone-introduction adjacent thereto. For example, the length may be 30 b to 300 b, preferably 40 b to 200 b, and more preferably 50 b to 100 b.

With the use of the helper plasmid for transformation according to the present invention, as described above, a plurality of linear nucleic acid fragments for gemone-introduction can be arranged in series and incorporated into the host genome. Each of the plurality of linear nucleic acid fragments for gemone-introduction may comprise a gene of interest, or some linear nucleic acid fragments for gemone-introduction may selectively comprise a gene of interest.

The transformation method and the method of producing a transformant using the helper plasmid for transformation according to the present invention are not particularly limited, and these methods can be applied to all types of host cells. Examples of the host cells include: fungi such as filamentous fungi and yeasts; bacteria such as Escherichia coli and Bacillus subtilis; plant cells; and animal cells including mammals and insects. Use of yeast host cells is particularly preferable. The type of the yeast is not particularly limited, and examples thereof include yeasts belonging to the genus Saccharomyces, yeasts belonging to the genus Kluyveromyces, yeasts belonging to the genus Candida, yeasts belonging to the genus Pichia, yeasts belonging to the genus Schizosaccharomyces, and yeasts belonging to the genus Hansenula. More specifically, the aforementioned methods can be applied to yeasts belonging to the genus Saccharomyces such as Saccharomyces cerevisiae, Saccharomyces bayanus, or Saccharomyces boulardii.

In the method of transformation and the method of producing a transformant using the helper plasmid for transformation according to the present invention, in particular, the helper plasmid for transformation comprises a counter selection marker. The counter selection marker can function to induce death of a host cell in which the linear nucleic acid fragment for gemone-introduction comprising a gene of interest remains incorporated in the helper plasmid for transformation. As shown in FIG. 3 or 5, when the helper plasmid for transformation according to the present invention is used, there is a case that the gene of interest may not be incorporated into genome DNA. The gene of interest may be present in the form of a circular helper plasmid for transformation in the host cell. If the helper plasmid for transformation does not comprise a counter selection marker and a transformed cell is selected based on the expression of the gene of interest or the selection marker introduced together with the gene of interest, a cell in which the gene of interest is not incorporated into genome DNA but is present in the form of a circular helper plasmid for transformation is selected (false-positive cell).

When the linear nucleic acid fragment for gemone-introduction comprising a gene of interest is cleaved by an endonuclease, as shown in FIG. 2 or 4, a helper plasmid for transformation becomes linearized. Thus, the helper plasmid would not be replicated and detached as the cell grows. When the linear nucleic acid fragment for gemone-introduction comprising a gene of interest is cleaved by the endonuclease, accordingly, a positive cell can be selected based on the expression of the gene of interest or a selection marker introduced together with the gene of interest without the influence of the counter selection marker.

### Second Embodiment

Hereafter, an embodiment in which a pair of endonuclease target sequences is provided in a linear nucleic acid fragment for gemone-introduction comprising a gene of interest is described. In the following description, the terms used in the first embodiment are used to omit detailed descriptions concerning the constitutions or the like.

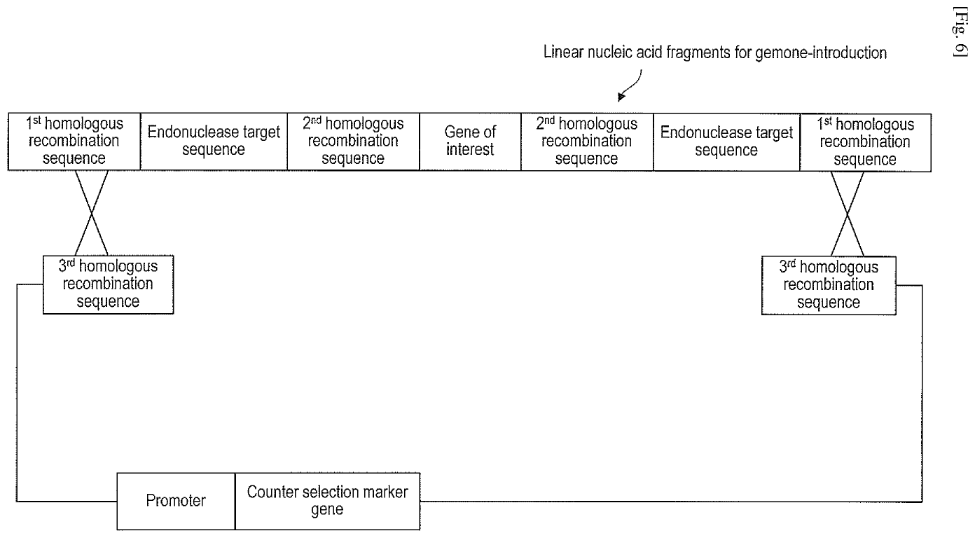

In the second embodiment, the linear nucleic acid fragment for gemone-introduction comprises: as shown in FIG. 6, a pair of the 1^{st} homologous recombination sequences capable of homologous recombination with the helper plasmid for transformation at the both ends; an endonuclease target sequence inside the 1^{st} homologous recombination sequences; a pair of the 2^{nd} homologous recombination sequences capable of homologous recombination with the host genome inside the endonuclease target sequence; and a gene of interest inside the pair of the 2^{nd} homologous recombination sequences. In the second embodiment, the helper plasmid for transformation comprises: a pair of the 3^{rd} homologous recombination sequences used for incorporation of the linear nucleic acid fragment for gemone-introduction; a promoter; and a counter selection marker gene downstream of the promoter. The helper plasmid for transformation may comprise an inducible promoter and an endonuclease gene downstream of the inducible promoter as shown in FIG. 4 in the first embodiment, although such configuration is not shown in FIG. 6.

In the second embodiment, a pair of the 3^{rd} homologous recombination sequences of the helper plasmid for transformation may undergo homologous recombination directly with a pair of the 1^{st} homologous recombination sequences of the linear nucleic acid fragment for gemone-introduction or homologous recombination indirectly via 1 or more linear nucleic acid fragments. Here, 1 or more linear nucleic acid fragments refer to one nucleic acid fragment or a nucleic acid fragment comprising a plurality of nucleic acid fragments linked to one another via homologous recombination, in which each comprise a sequence that can undergo homologous recombination with the 3^{rd} homologous recombination sequence of the helper plasmid for transformation at one end and a sequence that can undergo homologous recombination with the 1^{st} homologous recombination sequence of the linear nucleic acid fragment for gemone-introduction at the other end.

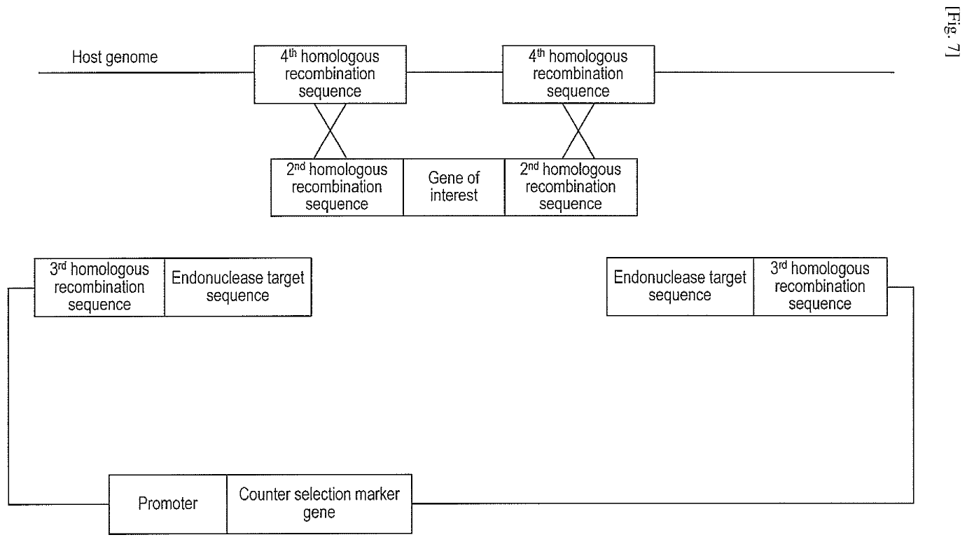

With the use of the linear nucleic acid fragment for gemone-introduction and the helper plasmid for transformation configured as described above, a stable transformant comprising a gene of interest incorporated into the genome can be produced in a simple and efficient manner. In order to produce a transformant, at the outset, the linear nucleic acid fragment for gemone-introduction comprising a gene of interest and the helper plasmid for transformation are introduced into the host cell in accordance with a conventional technique. In this case, homologous recombination takes place between the 1^{st} homologous recombination sequence of the linear nucleic acid fragment for gemone-introduction and the 3^{rd} homologous recombination sequence of the helper plasmid for transformation, and a circular plasmid comprising the linear nucleic acid fragment for gemone-introduction incorporated into the helper plasmid for transformation is produced (see FIG. 7). Thereafter, as schematically shown in FIG. 7, double strands of the pair of endonuclease target sequences are cleaved by the endonuclease, and a fragment comprising a gene of interest sandwiched with a pair of the 2^{nd} homologous recombination sequences is cleaved. The cleaved fragment undergoes crossing with the host genome at a site between a pair of the 2^{nd} homologous recombination sequence of the fragment and the 4^{th} homologous recombination sequence of the host genome, and the fragment is incorporated into the genome. Thus, a stable transformant comprising a gene of interest incorporated in the genome can be produced.

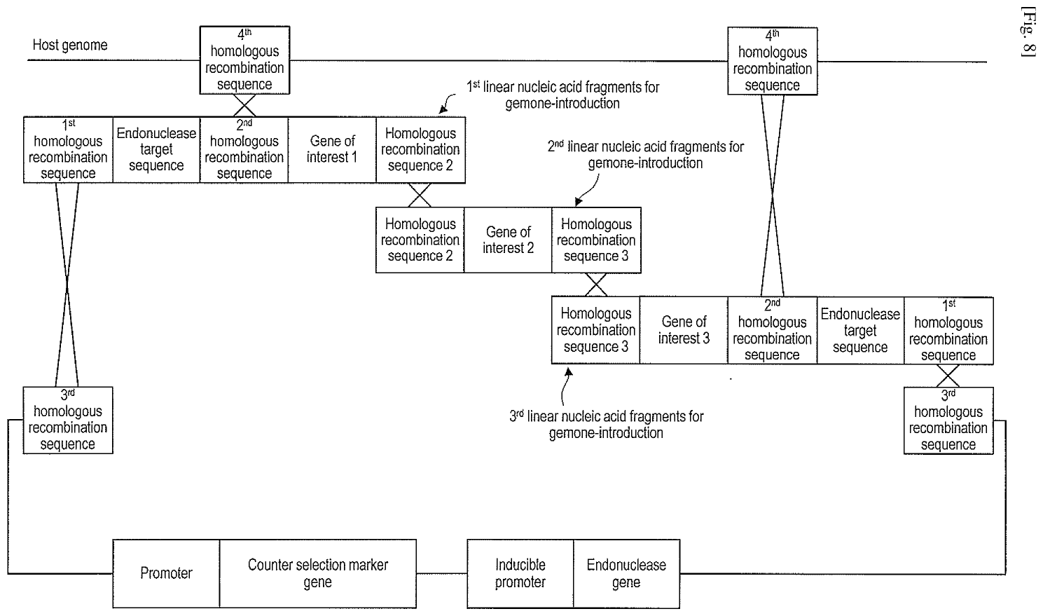

In the present embodiment, a plurality of linear nucleic acid fragments for gemone-introduction can be arranged in series and incorporated into the host genome. As shown in FIG. 8, when the 1^{st} to the 3^{rd} linear nucleic acid fragments for gemone-introduction are incorporated into the host genome, for example, the 3′ terminal sequence of the 1^{st} linear nucleic acid fragment for gemone-introduction and the 5′ terminal sequence of the 2^{nd} linear nucleic acid fragment for gemone-introduction are to be the homologous recombination sequence 2, and the 3′ terminal sequence of the 2^{nd} linear nucleic acid fragment for gemone-introduction and the 5′ terminal sequence of the 3^{rd} linear nucleic acid fragment for gemone-introduction are to be the homologous recombination sequence 3. Thus, the 1^{st} to the 3^{rd} linear nucleic acid fragments for gemone-introduction can be connected to one another in this order via homologous recombination. The obtained fragment is incorporated into the helper plasmid for transformation via homologous recombination between the 1^{st} homologous recombination sequence of the fragment and the 3^{rd} homologous recombination sequence of the helper plasmid for transformation. Also, a fragment cleaved at the endonuclease target sequences is incorporated into the host genome via homologous recombination between the 2^{nd} homologous recombination sequence of the fragment and the 4^{th} homologous recombination sequence of the genome.

According to the transformation method and the method of producing a transformant involving the use of the helper plasmid for transformation demonstrated as the second embodiment, also, the helper plasmid for transformation comprises a counter selection marker. The counter selection marker functions to induce death of the host in a manner such that the linear nucleic acid fragment for gemone-introduction comprising a gene of interest remains uncleaved but remains incorporated in the helper plasmid for transformation. As shown in FIG. 6 or 8, when the helper plasmid for transformation according to the present invention is used, there is a case that the gene of interest may not be incorporated into genome DNA, but the gene of interest may be present in the form of a circular helper plasmid for transformation in the host cell. If the helper plasmid for transformation does not comprise a counter selection marker and a transformed cell is selected based on the expression of the gene of interest or the selection marker introduced together with the gene of interest, a cell in which the gene of interest is not incorporated into genome DNA but is present in the form of a circular helper plasmid for transformation would also be selected (false-positive cell).

When the linear nucleic acid fragment for gemone-introduction comprising a gene of interest is cleaved by endonuclease, as shown in FIG. 7, the helper plasmid for transformation becomes linearized. Thus, the helper plasmid would not be replicated and detached as the cell grows. When the linear nucleic acid fragment for gemone-introduction comprising a gene of interest is cleaved by endonuclease, accordingly, a positive cell is selected based on the expression of the gene of interest or the selection marker introduced together with the gene of interest without the influence of the counter selection marker.

### EXAMPLES

Hereinafter, the present invention will be described in greater detail with reference to the following examples. However, these examples are not intended to limit the technical scope of the present invention.

### Example 1

Method

1. Test Strain

A monoploid experimental yeast, S. cerevisiae BY4742, was used as a test yeast line.

2. Production of a Plasmid Comprising a Linear Vector for ADE1 Disruption and a Helper Plasmid for Transformation

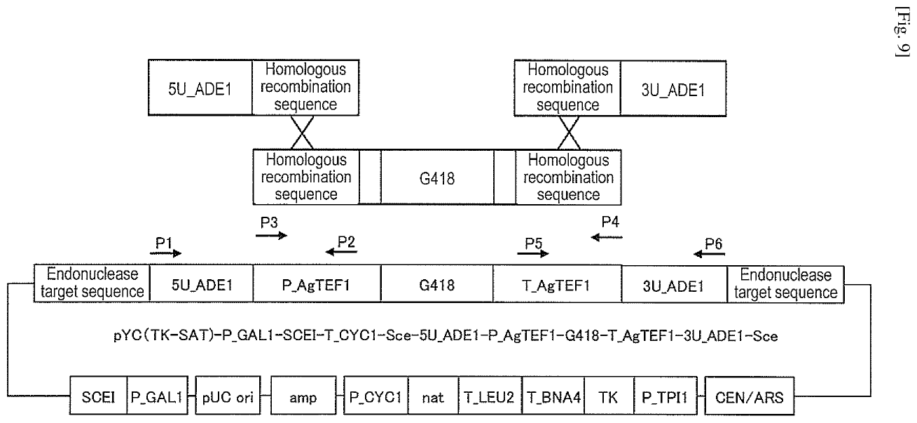

The plasmid produced was the YCp-type yeast shuttle vector pYC(TK-SAT)-P_GAL1-SCEI-T_CYC1-Sce-5U_ADE1-P_AgTEF1-G418-T_AgTEF 1-3U_ADE1-Sce comprising S. cerevisiae-derived homing endonuclease I-SceI induced by galactose (SCEI gene); a thymidine kinase as a counter selection marker; and a sequence formed by inserting a DNA fragment comprising homologous recombination sequences to be introduced into the genome between two recognition sequences of I-SceI (FIG. 9). pYC (TK-SAT)-P_GAL1-SCEI-T_CYC1-Sce-5U_ADE1-P_AgTEF1-G418-T_AgTEF1-3U_ADE1-Sce comprises a SCEI gene to which a GAL1 promoter and a CYC1 terminator had been added (a sequence into which the intron of a COX5B gene had been inserted, and in which codons in the whole length had been converted depending on the codon use frequency in the nuclear genome of the yeast), the herpes simplex virus type 1 thymidine kinase gene to which a TPI1 promoter and a BNA4 terminator had been added (a sequence in which codons in the whole length had been converted depending on the codon use frequency in the nuclear genome of the yeast; “TK” in FIG. 9), as homologous recombination sequences to be introduced into the genome, the gene sequence in a region approximately 1000 bp upstream of the 5′-terminal side of an ADE1 gene (5U_ADE1) and the DNA sequence in a region approximately 950 bp downstream of the 3′-terminal side of the ADE1 gene (3U_ADE1), and as a marker gene for homologous recombination, a gene sequence comprising a G418 resistance gene (G418 marker) to which Ashbya gossypii-derived TEF1 promoter and TEF1 terminator had been added. 5U_ADE1, 3U_ADE1, and the G418 marker sequence are inserted into a region between a pair of homing endonuclease I-SceI recognition sequences, and, therefore, can be cleaved with the aid of the SCEI gene added to the GAL1 promoter that is induced in a medium containing galactose as a carbon source.

Individual DNA sequences can be amplified by PCR. To connect individual DNA fragments to one another, primers were synthesized to have DNA sequence so as to overlap DNA sequence adjacent thereto by approximately 15 (Table 1). Using these primers, a DNA fragment of interest was amplified with the use of pRS436cen(SAT)-P_GAL1-SCEI-T_CYC1-Sce-5U_ADE1-P_AgTEF1-G418-T_AgT EF1-3U_ADE1-Sce (see, Reference Example below), the genome of the S. cerevisiae OC-2 strain, or synthetic DNA as a template, and the DNA fragments were connected to one another using the In-Fusion HD Cloning Kit or the like to produce a plasmid of interest.

TABLE 1

| Amplified DNA | Primer sequence | SEQ ID |
|---|---|---|
| fragment | (5′-3′) | NO: |
| TPI1 promoter | GGCAAGCGATCCGTCCTAG | 1 |
| | GCAAGAGAGAAGACCCAGA | |
| | GATGTTG | |
| | AGGATAACTGGCCATTTTT | 2 |
| | AGTTTATGTATGTGTTTTT | |
| | TGTAGTTATAGATTTAAG | |
| TK gene | ATGGCCAGTTATCCTTGTC | 3 |
| | ACC | |
| | TCAATTAGCTTCCCCCATT | 4 |
| | TCTC | |
| BNA4 | GGGGAAGCTAATTGAGAGCC | 5 |
| terminator | AGTTTATTCTTGCCATCC | |
| | TGAAACTATGATTCCTCGAT | 6 |
| | CAATGCGAAATTCCAACTAT | |
| | TTC | |
| Sequences | AGGAATCATAGTTTCATGAT | 7 |
| other than | TTTCTGTTAC | |
| TPI1 promoter, | GGACGGATCGCTTGCCTGTA | 8 |
| TK gene, | AC | |
| and BNA4 | | |
| terminator | | |

3. Production of a Linear DNA Vector Fragment for ADE1 Disruption

In this example, a fragment comprising the 5′ homologous recombination sequence of the ADE1 gene, a fragment comprising the 3′ homologous recombination sequence of the ADE1 gene, and a fragment comprising a G418 marker were amplified by PCR using the YCp-type yeast shuttle vector pYC (TK-SAT)-P_GAL1-SCEI-T_CYC1-Sce-5U_ADE1-P_AgTEF1-G418-T_AgTEF1-3U_ADE1-Sce produced above as a template and the primers shown in Table 2. More specifically, as schematically shown in FIG. 9, a fragment comprising the 5′ homologous recombination sequence of the ADE1 gene was amplified using the primers P1 and P2, a fragment comprising a G418 marker was amplified using the primers P3 and P4, and a fragment comprising the 3′ homologous recombination sequence of the ADE1 gene was amplified using the primers P5 and P6. Primers were designed, so that fragments would overlap with each other by approximately 60 bp.

TABLE 2

| Amplified | | Primer | SEQ |
|---|---|---|---|
| DNA | | sequence | ID |
| fragment | Primer | (5′-3′) | NO: |
| Fragment | P1 | ACGGATTAGAA | 9 |
| comprising | | GCCGCCGAG | |
| ADE1 | P2 | TCATGCCCCTG | 10 |
| 5′ | | AGCTGCG | |
| homologous | | | |
| recombination | | | |
| sequence | | | |
| Fragment | P3 | GACATGGAGGC | 11 |
| comprising | | CCAGAATAC | |
| G418 | P4 | CAGTATAGCGA | 12 |
| marker | | CCAGCATTC | |
| Fragment | P5 | TTAAGTGCGCA | 13 |
| comprising | | GAAAGTAAT | |
| ADE1 | | ATCATG | |
| 3′ | P6 | TGACCGGATGA | 14 |
| homologous | | AACCACC | |
| recombination | | | |
| sequence | | | |

4. Production of a Helper Plasmid for Transformation

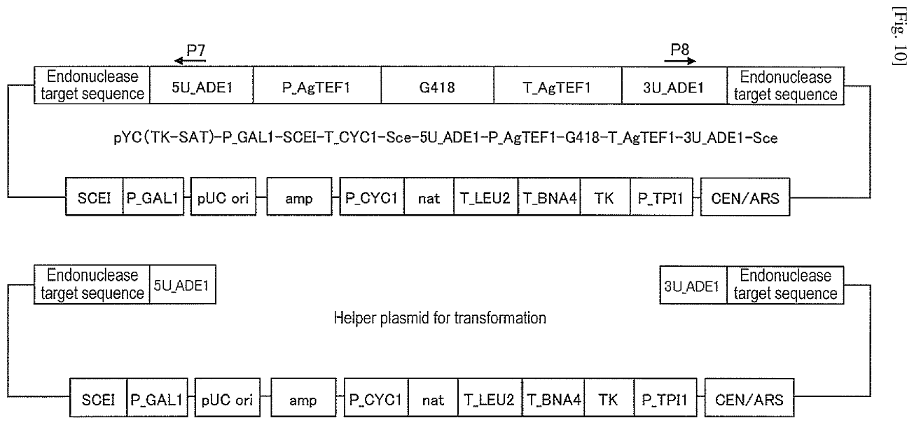

In this example, a helper plasmid for transformation was amplified using the YCp-type yeast shuttle vector pYC (TK-SAT)-P_GAL1-SCEI-T_CYC1-Sce-5U_ADE1-P_AgTEF1-G418-T_AgTEF1-3U_ADE1-Sce produced above as a template and the primers shown in Table 3, where the helper plasmid for transformation overlap with the linear nucleic acid fragment for gemone-introduction comprising the 5′ or 3′ homologous recombination sequence of the ADE1 gene by approximately 60 bp. More specifically, as schematically shown in FIG. 10, a helper plasmid for transformation was amplified by PCR using the primers P7 and P8.

TABLE 3

| | | Primer | SEQ |
|---|---|---|---|
| Amplified DNA | | sequence | ID |
| fragment | Primer | (5′-3′) | NO: |
| Helper plasmid | P7 | GGTTTCAGATCA | 15 |
| for transformation | | CGATGGATAAC | |
| | P8 | GCAACAGTAAAA | 16 |
| | | GGGATCAGC | |

5. Acquisition of an ADE1-Disrupted Strain Via Simultaneous Transformation of a Linear DNA Vector for ADE1 Disruption and a Helper Plasmid for Transformation

The linear DNA vector for ADE1 disruption and the helper plasmid for transformation produced were used (400 fmol each) to transform the S. cerevisiae BY4742 strain, culture was conducted in a YPD liquid medium for 4 hours, the culture product was applied to a G418-containing YPGa (carbon source: galactose) agar medium (2×10⁶ cells/plate), and grown colonies were then counted. At the same time, the culture product was applied to a YPD agar medium (2×10⁶ cells/plate), and the number of actually grown colonies was determined as the number of cells sowed in the agar medium. Transformation was carried out according to the method of Akada et al. (Akada, R. et al., “Elevated temperature greatly improves transformation of fresh and frozen competent cells in yeast,” BioTechniques 28, 2000: 854-856).

The ADE1 gene is a gene of adenine biosynthesis pathway. In the ADE1 gene-disrupted strain, 5-aminoimidazole riboside as an intermediate metabolite of adenine is accumulated, and the polyribosylaminoimidazole polymerized therewith is colored red. Thus, the ADE1 gene-disrupted strain can be easily distinguished. Efficiency of homologous recombination into the ADE1 gene locus was calculated in accordance with the following equation.

ADE1 gene disruption frequency=the number of red colonies grown in agar medium/the number of cells sowed in agar medium

6. Production of a Strain Comprising a Circular Fusion Vector of a Linear DNA Vector for ADE1 Disruption and a Helper Plasmid for Transformation Introduced Therein and Acquisition of an ADE1-Disrupted Strain

In this example, for comparison, cells directly transformed from pYC(TK-SAT)-P_GAL1-SCEI-T_CYC1-Sce-5U_ADE1-P_AgTEF1-G418-T_AgTEF 1-3U_ADE1-Sce were cultured in a YPD liquid medium for 4 hours, the culture product was applied to a nourseothricin-containing YPD agar medium, and grown colonies were determined as the strains into each of which a circular fusion vector of a linear DNA vector for ADE1 disruption and a helper plasmid for transformation had been introduced. The vector-introduced strains were transferred to a G418-containing YPGa medium, and the linear DNA vector for ADE1 disruption was cleaved to obtain ADE1-disrupted strains on the genome.

7. Removal of a Helper Plasmid for Transformation Via Counter Selection

A thymidine kinase gene encodes an enzyme that converts 5-fluoro-2-deoxyuridine (5FU) into a toxic metabolite. Accordingly, a cell comprising a thymidine kinase gene is induced to die in a 5FU-containing medium. Specifically, cells from which the helper plasmid for transformation having the thymidine kinase gene have been detached can selectively grow. Among the colonies grown in a G418-containing YPGa agar medium, 30 white colonies and 10 red colonies were transferred to a 5FU-containing SD agar medium, and the ADE1 disruption rate and the viability were determined based on the colony color. On the basis of the calculation above, a false-positive rate after counter selection was calculated in accordance with the following equation.

False-positive rate after counter selection=putative number of white colonies after counter selection/putative number of colonies survived after counter selection

### Results and Discussion

1. Effects of Helper DNA on Genome Transformation Efficiency of Circular DNA Plasmid

When a linear DNA vector and a helper plasmid for transformation are simultaneously added, the efficiency for obtaining ADE1-disrupted strains was approximately 5 times greater than that attained when a circular fusion vector containing a linear DNA vector was added by itself (Table 4). However, ADE1-undisrupted white colonies were obtained at high frequency (Table 4).

TABLE 4

| | A circular fusion | A linear vector for |
|---|---|---|
| | plasmid comprising | ADE1 disruption + |
| | a linear vector for | a helper plasmid |
| | ADE1 disruption | for transformation |
| ADE1 disruption frequency | 8.2 × 10⁻⁵ | 4.9 × 10⁻⁴ |
| (G418 resistant and red | | |
| colonies) | | |
| False-positive frequency | 4.2 × 10⁻⁶ | 1.7 × 10⁻⁴ |
| (G418 resistant and white | | |
| colonies) | | |

Subsequently, transformed colonies obtained by simultaneous transformation of the linear DNA vector for ADE1 disruption and the helper plasmid for transformation were transferred to a 5FU medium and subjected to counter selection. The results are shown in Table 5.

TABLE 5

| | Red | White | Dead |
|---|---|---|---|
| | colonies | colonies | colonies |
| ADE1-disrupted | 100% | 0% | 0% |
| clone-derived | | | |
| (red colonies) | | | |
| False-positive | 16.7% | 3.3% | 80.0% |
| clone-derived | | | |
| (white colonies) | | | |

As shown in Table 5, all the transformed ADE1-disrupted strains (red colonies) grew as red colonies; however, phenotypes of ADE1-undisrupted strains (white colonies) were divided into 3 types: red colonies, white colonies, and dead colonies. When colonies resulting from introduction of a circular fusion plasmid containing a linear vector for ADE1 disruption were transferred to a counter selection medium, no particular change was observed in the phenotype.

It is considered that dead colonies had acquired G418 resistance for the following reasons. That is, such dead colonies retain plasmids in which, for some reasons, linear vectors were not cleaved from the circular plasmids resulting from homologous recombination between the linear DNA vector and the helper plasmid for transformation and thus have acquired G418 resistance. It is considered that the cells retaining such plasmids were induced to die as a result of counter selection and that cells from which such plasmids had been detached acquired G418 sensitivity. Colonies that had turned red are considered to result from the presence of a very small number of ADE1-disrupted cells in white colonies that were considered to be ADE1-undisrupted strains. It is considered that ADE1-disrupted strains have preferentially grown as a result of counter selection. As shown in Table 5, it was found that, as a result of counter selection, there would be substantially no false-positive cells that retain G418 resistance while remaining in the form of white colonies.

Table 6 shows the results of comparison of the false-positive rate after counter selection between the introduction of a circular fusion plasmid comprising a linear vector for ADE1 disruption and simultaneous introduction of a linear DNA vector and a plasmid for transformation.

TABLE 6

| | A circular fusion | A linear vector for |
|---|---|---|
| | plasmid comprising | ADE1 disruption + |
| | a linear vector for | a helper plasmid |
| | ADE1 disruption | for transformation |
| ADE1 disruption frequency | 8.2 × 10⁻⁵ | 5.2 × 10⁻⁴ |
| (G418 resistant and red | | |
| colonies) | | |
| False-positive frequency | 4.2 × 10⁻⁶ | 5.6 × 10⁻⁶ |
| (G418 resistant and white | | |
| colonies) | | |
| False-positive rate | 4.8 × 10⁻² | 1.1 × 10⁻² |

Upon simultaneous introduction of the linear DNA vector and the helper plasmid for transformation, as shown in Table 6, a false-positive rate became lower than that attained upon introduction of a circular fusion plasmid containing a linear vector for ADE1 disruption. The results demonstrate that removal of false-positive clones by the methods of the present invention are very effective.

### Reference Example

pRS436cen(SAT)-P_GAL1-SCEI-T_CYC1-Sce-5U_ADE1-P_AgTEF1-G418-T_Ag TEF1-3U_ADE1-Sce is a vector in which a 2-microM plasmid-derived replication origin is deleted from the pRS436(SAT)-P_GAL1-SCEI-T_CYC1-Sce-5U_ADE1-P_AgTEF1-G418-T_AgTEF1-3U_ADE1-Sce vector and, instead thereof, an autonomous replication sequence (ARS) and a centromere sequence (CEN) are inserted therein. The copy number in a cell is retained to be one. This vector was produced by amplifying a DNA fragment of interest using RS436(SAT)-P_GAL1-SCEI-T_CYC1-Sce-5U_ADE1-P_AgTEF1-G418-T_AgTEF1-3U_ADE1-Sce or the genome of the S. cerevisiae OC-2 strain as a template (the primers used are shown in Table 7) and binding the DNA fragments to one another using the In-Fusion Kit or the like.

pRS436(SAT)-P_GAL1-SCEI-T_CYC1-Sce-5U_ADE1-P_AgTEF1-G418-T_AgTE F1-3U_ADE1-Sce comprises: a SCEI gene to which a GAL1 promoter and a CYC1 terminator had been added (a sequence into which the intron of a COX5B gene had been inserted, and in which codons in the whole length had been converted depending on the codon use frequency in the nuclear genome of the yeast); a gene sequence comprising a nourseothricin resistance gene (nat marker); as homologous recombination sequences to be introduced into the genome, the gene sequence in a region approximately 1000 bp upstream of the 5′-terminal side of an ADE1 gene (5U_ADE1) and the DNA sequence in a region approximately 950 bp downstream of the 3′-terminal side of the ADE1 gene (3U_ADE1); and as a marker gene for homologous recombination, a gene sequence comprising a G418 resistance gene (G418 marker) to which Ashbya gossypii-derived TEF1 promoter and TEF1 terminator had been added, inserted into a vector prepared by removing a URA3 gene, a TDH3 promoter, and a CYC1 terminator from the YEp-type yeast shuttle vector pRS436GAP (NCBI Accession Number: AB304862). 5U_ADE1, 3U_ADE1, and the G418 marker sequence are inserted into a region between a pair of homing endonuclease I-SceI recognition sequences, and such region can be cleaved by the SECI gene added to the GAL1 promoter inducible in a medium containing galactose as a carbon source.

Individual DNA sequences can be amplified by PCR. To connect individual DNA fragments to one another, primers were synthesized to have DNA sequence so as to overlap DNA sequence adjacent thereto by approximately 15 (Table 7). Using these primers, a DNA fragment of interest was amplified with the use of the genome of the S. cerevisiae OC-2 strain or synthetic DNA as a template, and the DNA fragments were successively connected to one another using the In-Fusion HD Cloning Kit or the like. The resultant was cloned into the pRS436GAP vector to produce a final plasmid of interest.

TABLE 7

| | | |
|---|---|---|
| Amplified DNA | | SEQ |
| fragment | Primer sequence (5′-3′) | ID NO: |

pRS436(SAT)- P_GAL1-SCEI-T_CYC1-Sce-5U_ADE1-P_AgTEF1-G418-T_AgTEF1-3U_ADE1-Sce

| | | |
|---|---|---|
| GAL1 promoter | ACGGATTAGAAGCCGCCGAG | 17 |
| | GGTTTTTTCTCCTTGACGTTAAAGTATAG | 18 |
| COX5B intron | TCAAGGAGAAAAAACCAGCATGTATAACAAACACTGATTTTTGTTTTG | 19 |
| | TCTTAATGTTTTTCACTGCAAAACTTGTGCTTGTACAC | 20 |
| SCEI | TGAAAAACATTAAGAAAAACCAAGTTATG | 21 |
| | GCGTGACATAACTAATCATTTCAAGAAGGTTTCGGAG | 22 |
| CYC1 terminator | TTAGTTATGTCACGCTTACATTCACG | 23 |
| (comprising I-SceI | AATTGCCCGACTCATATTACCCTGTTATCCCTAAGCTTGCAAATTA | 24 |
| target sequence) | AAGCCTTCGAGCG | |
| 5U_ADE1 | ATGAGTCGGGCAATTCCG | 25 |
| | CTGGGCCTCCATGTCTATCGTTAATATTTCGTATGTGTATTCTTTG | 26 |
| TEF1 promoter | GACATGGAGGCCCAGAATAC | 27 |
| derived from | GGTTGTTTATGTTCGGATGTGATG | 28 |
| Ashbya gossypii | | |
| G418 | CGAACATAAACAACCATGGGTAAGGAAAAGACTCACGTTTC | 29 |
| | TATTGTCAGTACTGATTAGAAAAACTCATCGAGCATCAAATGAAAC | 30 |
| TEF1 terminator | TCAGTACTGACAATAAAAAGATTCTTGTTTTCAAG | 31 |
| derived from | CAGTATAGCGACCAGCATTCACATACG | 32 |
| Ashbya gossypii | | |
| TEF1 promoter | ATAGCATACATTATACGAAGTTATCCCACACACCATAGCTTCAAAATG | 33 |
| (comprising part of | CACCGAAATCTTCATCCCTTAGATTAGATTGCTATGC | 34 |
| LoxP sequence) | | |
| 3U_ADE1 | GCTGGTCGCTATACTGCGTGATTTACATATACTACAAGTCG | 35 |
| (comprising I-SceI | AAAAACATAAGACAAATTACCCTGTTATCCCTATGACCGGATGAAACC | 36 |
| target sequence) | | |
| pRS436 | GGGATAACAGGGTAATGGTACCCAATTCGCCCTATAG | 37 |
| (comprising 2 μ | TACCGCACAGATGCGTAAGG | 38 |
| replication origin) | | |
| LEU2 terminator | TTACGCATCTGTGCGGTAAGGAATCATAGTTTCATGATTTTCTG | 39 |
| | CAGGATGACGCCTAAAAAGATTCTCTTTTTTTATGATATTTGTAC | 40 |
| nourseothricin | TTAGGCGTCATCCTGTGCTC | 41 |
| resistance gene | CACACTAAATTAATAATGAAGATTTCGGTGATCCC | 42 |
| CYC1 promoter | TATTAATTTAGTGTGTGTATTTGTGTTTGTGTG | 43 |
| | GCAGATTGTACTGAGAGTACGACATCGTCGAATATGATTC | 44 |
| pRS436 | ACTCTCAGTACAATCTGCTCTGATGC | 45 |
| (comprising an | CGGCTTCTAATCCGTGCTCCAGCTTTTGTTCCCTTTAG | 46 |
| ampicillin | | |
| resistance gene and | | |
| ColE1 replication | | |
| origin) | | |

pRS436cen(SAT)-P_GAL1-SCEI-T_CYC1-Sce-5U_ADE1-P_AgTBF1-G418-T_AgTEF1-3U_ADE1-Sce

| | | |
|---|---|---|
| Sequence other than | TCAAGGAGAAAAAACCAGCATGTATAACAAACACTGATTTTTGTTTTG | 47 |
| GAL1 promoter | CGGCTTCTAATCCGTGCTCCAGCTTTTGTTCCCTTTAG | 48 |
| GAL1 promoter | GGTCCTTTTCATCACGTGCTA | 49 |
| | GGTTTTTTCTCCTTGACGTTAAAGTATAG | 50 |

All publications, patents, and patent applications cited herein are incorporated herein by reference in their entirety.

1. A method of producing a transformant comprising:
a step of introducing: one or more linear nucleic acid fragments for gemone-introduction comprising a gene of interest to be introduced into a given site of a genome; and a helper plasmid for transformation comprising a pair of homologous recombination sequences to incorporate the linear nucleic acid fragment and a counter selection marker, into a host, wherein, in a state of introducing the linear nucleic acid fragment into the helper plasmid for transformation, a pair of homologous recombination sequences that undergoes homologous recombination between a region outside of the gene of interest and a given site of the genome and a pair of endonuclease target sequences at the outside of the pair of homologous recombination sequences are provided; and
a step of selecting a transformant in which the gene of interest is incorporated into the given site of the host genome, and in which the gene of interest is expressed,
wherein the counter selection marker functions to induce the death of a host comprising the helper plasmid for transformation into which the linear nucleic acid fragment is incorporated.

2. The method of producing a transformant according to claim 1, wherein the helper plasmid for transformation comprises: a pair of homologous recombination sequences that undergoes homologous recombination with regions outside of a gene of interest in the linear nucleic acid fragment for gemone-introduction; and a pair of endonuclease target sequences provided on the other side of a site into which the linear nucleic acid fragment for gemone-introduction is incorporated via the homologous recombination sequences.

3. The method of producing a transformant according to claim 1, wherein the linear nucleic acid fragment for gemone-introduction comprises: the pair of homologous recombination sequences to be incorporated into a given site of the genome at positions sandwiching the gene of interest; the pair of endonuclease target sequences outside of the pair of homologous recombination sequences; and the pair of homologous recombination sequences that undergoes homologous recombination with the helper plasmid for transformation outside of the pair of endonuclease target sequences.

4. The method of producing a transformant according to claim 1, wherein the helper plasmid for transformation comprises a target-specific endonuclease gene specifically cleaving the double strands of the endonuclease target sequences in an expressible manner.

5. The method of producing a transformant according to claim 4, wherein the target-specific endonuclease gene is a homing endonuclease gene.

6. The method of producing a transformant according to claim 5, wherein the endonuclease target sequence is specifically recognized by homing endonuclease.

7. The method of producing a transformant according to claim 4, wherein the helper plasmid for transformation comprises an inducible promoter regulating the expression of the target-specific endonuclease gene.

8. The method of producing a transformant according to claim 1, wherein the plurality of the linear nucleic acid fragments consist of a 1^{st} linear nucleic acid fragment for gemone-introduction to the n^{th} linear nucleic acid fragment for gemone-introduction (n: an integer of 2 or greater), and a 3′ terminal sequence of the m^{th} linear nucleic acid fragment for gemone-introduction (m: an integer that satisfies the correlation: 1<=m<=n−1) comprises a sequence that undergoes homologous recombination with a 5′ terminal sequence of the m^{th}+1 linear nucleic acid fragment for gemone-introduction.

9. A transformation method comprising:
a step of introducing: one or more linear nucleic acid fragments for gemone-introduction comprising a gene of interest to be introduced into a given site of a genome; and a helper plasmid for transformation comprising a pair of homologous recombination sequences to incorporate the linear nucleic acid fragment and a counter selection marker, into a host, wherein, in a state of introducing the linear nucleic acid fragment into the helper plasmid for transformation, a pair of homologous recombination sequences that undergoes homologous recombination between a region outside of the gene of interest and a given site of the genome and a pair of endonuclease target sequences at the outside of the pair of homologous recombination sequences are provided,
wherein the counter selection marker functions to induce the death of a host comprising the helper plasmid for transformation into which the linear nucleic acid fragment is incorporated.

10. The transformation method according to claim 9, wherein the helper plasmid for transformation comprises: a pair of homologous recombination sequences that undergoes homologous recombination with regions outside of a gene of interest in the linear nucleic acid fragment for gemone-introduction; and a pair of endonuclease target sequences provided on the other side of a site into which the linear nucleic acid fragment for gemone-introduction is incorporated via the homologous recombination sequences.

11. The transformation method according to claim 9, wherein the linear nucleic acid fragment for gemone-introduction comprises: the pair of homologous recombination sequences to be incorporated into a given site of the genome at positions sandwiching the gene of interest; the pair of endonuclease target sequences outside of the pair of homologous recombination sequences; and the pair of homologous recombination sequences that undergoes homologous recombination with the helper plasmid for transformation outside of the pair of endonuclease target sequences.

12. The transformation method according to claim 9, wherein the helper plasmid for transformation comprises a target-specific endonuclease gene specifically cleaving the double strands of the endonuclease target sequences in an expressible manner.

13. The transformation method according to claim 12, wherein the target-specific endonuclease gene is a homing endonuclease gene.

14. The transformation method according to claim 13, wherein the endonuclease target sequence is specifically recognized by homing endonuclease.

15. The transformation method according to claim 12, wherein the helper plasmid for transformation comprises an inducible promoter regulating the expression of the target-specific endonuclease gene.

16. The transformation method according to claim 9, wherein the plurality of the linear nucleic acid fragments consist of a 1^{st} linear nucleic acid fragment for gemone-introduction to the n^{th} linear nucleic acid fragment for gemone-introduction (n: an integer of 2 or greater), and a 3′ terminal sequence of the m^{th} linear nucleic acid fragment for gemone-introduction (m: an integer that satisfies the correlation: 1<=m<=n−1) comprises a sequence that undergoes homologous recombination with a 5′ terminal sequence of the m^{th}+1 linear nucleic acid fragment for gemone-introduction.

17. A helper plasmid for transformation capable of incorporating a linear nucleic acid fragment for gemone-introduction comprising a gene of interest to be introduced into a given site of a genome via homologous recombination, which comprises a pair of homologous recombination sequences that undergoes homologous recombination with regions outside of a gene of interest in the linear nucleic acid fragment for gemone-introduction; a pair of endonuclease target sequences provided on the other side of a site into which the linear nucleic acid fragment for gemone-introductions is incorporated via the homologous recombination sequences; and a counter selection marker.

18. The helper plasmid for transformation according to claim 17, which comprises a target-specific endonuclease gene specifically cleaving the double strands of the endonuclease target sequences in an expressible state.

19. The helper plasmid for transformation according to claim 18, wherein the target-specific endonuclease gene is a homing endonuclease gene.

20. The helper plasmid for transformation according to claim 19, wherein the endonuclease target sequence is specifically recognized by homing endonuclease.

21. The helper plasmid for transformation according to claim 18, which comprises an inducible promoter regulating the expression of the target-specific endonuclease gene.


SEQUENCE LISTING (50 sequences)

SEQ ID NO: 1 - DNA, 45 bp, Artificial
  Synthetic DNA
ggcaagcgatccgtcctaggcaagagagaagacccagagatgttg

SEQ ID NO: 2 - DNA, 56 bp, Artificial
  Synthetic DNA
aggataactggccatttttagtttatgtatgtgttttttgtagttatagatttaag

SEQ ID NO: 3 - DNA, 22 bp, Artificial
  Synthetic DNA
atggccagttatccttgtcacc

SEQ ID NO: 4 - DNA, 23 bp, Artificial
  Synthetic DNA
tcaattagcttcccccatttctc

SEQ ID NO: 5 - DNA, 38 bp, Artificial
  Synthetic DNA
ggggaagctaattgagagccagtttattcttgccatcc

SEQ ID NO: 6 - DNA, 43 bp, Artificial
  Synthetic DNA
tgaaactatgattcctcgatcaatgcgaaattccaactatttc

SEQ ID NO: 7 - DNA, 30 bp, Artificial
  Synthetic DNA
aggaatcatagtttcatgattttctgttac

SEQ ID NO: 8 - DNA, 22 bp, Artificial
  Synthetic DNA
ggacggatcgcttgcctgtaac

SEQ ID NO: 9 - DNA, 20 bp, Artificial
  Synthetic DNA
acggattagaagccgccgag

SEQ ID NO: 10 - DNA, 18 bp, Artificial
  Synthetic DNA
tcatgcccctgagctgcg

SEQ ID NO: 11 - DNA, 20 bp, Artificial
  Synthetic DNA
gacatggaggcccagaatac

SEQ ID NO: 12 - DNA, 27 bp, Artificial
  Synthetic DNA
cagtatagcgaccagcattcacatacg

SEQ ID NO: 13 - DNA, 26 bp, Artificial
  Synthetic DNA
ttaagtgcgcagaaagtaatatcatg

SEQ ID NO: 14 - DNA, 18 bp, Artificial
  Synthetic DNA
tgaccggatgaaaccacc

SEQ ID NO: 15 - DNA, 23 bp, Artificial
  Synthetic DNA
ggtttcagatcacgatggataac

SEQ ID NO: 16 - DNA, 21 bp, Artificial
  Synthetic DNA
gcaacagtaaaagggatcagc

SEQ ID NO: 17 - DNA, 20 bp, Artificial
  Synthetic DNA
acggattagaagccgccgag

SEQ ID NO: 18 - DNA, 29 bp, Artificial
  Synthetic DNA
ggttttttctccttgacgttaaagtatag

SEQ ID NO: 19 - DNA, 48 bp, Artificial
  Synthetic DNA
tcaaggagaaaaaaccagcatgtataacaaacactgatttttgttttg

SEQ ID NO: 20 - DNA, 38 bp, Artificial
  Synthetic DNA
tcttaatgtttttcactgcaaaacttgtgcttgtacac

SEQ ID NO: 21 - DNA, 29 bp, Artificial
  Synthetic DNA
tgaaaaacattaagaaaaaccaagttatg

SEQ ID NO: 22 - DNA, 37 bp, Artificial
  Synthetic DNA
gcgtgacataactaatcatttcaagaaggtttcggag

SEQ ID NO: 23 - DNA, 26 bp, Artificial
  Synthetic DNA
ttagttatgtcacgcttacattcacg

SEQ ID NO: 24 - DNA, 59 bp, Artificial
  Synthetic DNA
aattgcccgactcatattaccctgttatccctaagcttgcaaattaaagccttcgagcg

SEQ ID NO: 25 - DNA, 18 bp, Artificial
  Synthetic DNA
atgagtcgggcaattccg

SEQ ID NO: 26 - DNA, 46 bp, Artificial
  Synthetic DNA
ctgggcctccatgtctatcgttaatatttcgtatgtgtattctttg

SEQ ID NO: 27 - DNA, 20 bp, Artificial
  Synthetic DNA
gacatggaggcccagaatac

SEQ ID NO: 28 - DNA, 24 bp, Artificial
  Synthetic DNA
ggttgtttatgttcggatgtgatg

SEQ ID NO: 29 - DNA, 41 bp, Artificial
  Synthetic DNA
cgaacataaacaaccatgggtaaggaaaagactcacgtttc

SEQ ID NO: 30 - DNA, 46 bp, Artificial
  Synthetic DNA
tattgtcagtactgattagaaaaactcatcgagcatcaaatgaaac

SEQ ID NO: 31 - DNA, 35 bp, Artificial
  Synthetic DNA
tcagtactgacaataaaaagattcttgttttcaag

SEQ ID NO: 32 - DNA, 27 bp, Artificial
  Synthetic DNA
cagtatagcgaccagcattcacatacg

SEQ ID NO: 33 - DNA, 48 bp, Artificial
  Synthetic DNA
atagcatacattatacgaagttatcccacacaccatagcttcaaaatg

SEQ ID NO: 34 - DNA, 37 bp, Artificial
  Synthetic DNA
caccgaaatcttcatcccttagattagattgctatgc

SEQ ID NO: 35 - DNA, 41 bp, Artificial
  Synthetic DNA
gctggtcgctatactgcgtgatttacatatactacaagtcg

SEQ ID NO: 36 - DNA, 48 bp, Artificial
  Synthetic DNA
aaaaacataagacaaattaccctgttatccctatgaccggatgaaacc

SEQ ID NO: 37 - DNA, 37 bp, Artificial
  Synthetic DNA
gggataacagggtaatggtacccaattcgccctatag

SEQ ID NO: 38 - DNA, 20 bp, Artificial
  Synthetic DNA
taccgcacagatgcgtaagg

SEQ ID NO: 39 - DNA, 44 bp, Artificial
  Synthetic DNA
ttacgcatctgtgcggtaaggaatcatagtttcatgattttctg

SEQ ID NO: 40 - DNA, 45 bp, Artificial
  Synthetic DNA
caggatgacgcctaaaaagattctctttttttatgatatttgtac

SEQ ID NO: 41 - DNA, 20 bp, Artificial
  Synthetic DNA
ttaggcgtcatcctgtgctc

SEQ ID NO: 42 - DNA, 35 bp, Artificial
  Synthetic DNA
cacactaaattaataatgaagatttcggtgatccc

SEQ ID NO: 43 - DNA, 33 bp, Artificial
  Synthetic DNA
tattaatttagtgtgtgtatttgtgtttgtgtg

SEQ ID NO: 44 - DNA, 40 bp, Artificial
  Synthetic DNA
gcagattgtactgagagtacgacatcgtcgaatatgattc

SEQ ID NO: 45 - DNA, 26 bp, Artificial
  Synthetic DNA
actctcagtacaatctgctctgatgc

SEQ ID NO: 46 - DNA, 38 bp, Artificial
  Synthetic DNA
cggcttctaatccgtgctccagcttttgttccctttag

SEQ ID NO: 47 - DNA, 48 bp, Artificial; identical to SEQ ID NO: 19
  Synthetic DNA

SEQ ID NO: 48 - DNA, 38 bp, Artificial; identical to SEQ ID NO: 46
  Synthetic DNA

SEQ ID NO: 49 - DNA, 21 bp, Artificial
  Synthetic DNA
ggtccttttcatcacgtgcta

SEQ ID NO: 50 - DNA, 29 bp, Artificial
  Synthetic DNA
ggttttttctccttgacgttaaagtatag